(19) United States
(12) Patent Application Publication
(10) Pub. No.: US 2018/0353280 A1
(43) Pub. Date: Dec. 13, 2018

(54) INFARCTION PREVENTION DEVICE AND TREATMENT METHOD

(71) Applicant: Dai YAMANOUCHI, Verona, WI (US)

(72) Inventor: Dai YAMANOUCHI, Verona, WI (US)

(21) Appl. No.: 16/006,259

(22) Filed: Jun. 12, 2018

Related U.S. Application Data
(60) Provisional application No. 62/518,724, filed on Jun. 13, 2017.

(51) Int. Cl. A61F 2/01

(52) U.S. Cl. CPC A61F 2/013; A61F 2002/015; A61F 2002/016

(57) ABSTRACT

An infarction prevention device has a filter to be placed inside a blood vessel. The filter includes a connection portion having a base end to be connected to a placing member configured to place the filter inside the blood vessel, and a cylindrical body portion connected to a front end of the connection portion. At least one of the connection portion and the cylindrical body portion includes a capturing portion configured to capture a foreign substance included in blood inside the blood vessel such that the at least one of the connection portion and the body portion controls a flow of the foreign substance.

### CROSS-REFERENCE TO RELATED APPLICATION

This application claims benefit to U.S. Provisional Application No. 62/518,724 filed on Jun. 13, 2017; the entire content of which is incorporated herein by reference.

### FIELD OF INVENTION

The present invention relates to an infarction prevention device and a treatment method.

### RELATED ART

During intravascular treatment, there is a concern that foreign substances such as thrombi are generated and dispersed inside the blood vessel to cause infarction. U.S. Pat. No. 8,182,507 discloses a vascular protection device including a filter attached to a distal end of an elongated member to be inserted into a blood vessel. The filter captures a foreign substance such as thrombi and prevents the foreign substances from flowing into an end artery, an end vein, and the like.

In a treatment of an aortic aneurysm, aortic dissection, and the like occurred inside an aorta using a stent graft, infarction may develop due to generated foreign substances such as thrombi, and therefore, technique for suitably preventing the infarction is required.

### SUMMARY

Illustrative aspects of the present invention provide an infarction prevention device and a treatment method capable of suitably preventing the onset of infarction.

According to an aspect of the present invention an infarction prevention device includes a filter to be placed inside a blood vessel. The filter includes a connection portion having a base end to be connected to placing member configured to place the filter inside the blood vessel, and a body portion having a cylindrical outer shape and connected to a front end of the connection portion. At least one of the connection portion and the body portion comprises a capturing portion configured to capture a foreign substance included in blood inside the blood vessel such that the at least one of the connection portion and the body portion controls a flow of the foreign substance included in the blood inside the blood vessel

According to another aspect of the present invention, a treatment method includes placing the filter of the infarction prevention device inside the blood vessel, and after the placing, treating another blood vessel different from the blood vessel using an intravascular treatment tool.

### BRIEF DESCRIPTION OF THE DRAWINGS

FIG. 1 is a view schematically illustrating a state in which a stent graft is placed in an aortic arch and an infarction prevention device according to the present invention is temporarily placed inside a brachiocephalic trunk;

FIGS. 2A and 2B are perspective views illustrating an infarction prevention device according to a first embodiment of the present invention, wherein FIG. 2A schematically illustrates a state in which a filter of the infarction prevention device is placed in position inside a blood vessel and FIG. 2B schematically illustrates a state in which the filter is moved inside the blood vessel;

FIG. 3 is a perspective view of the filter of the infarction prevention device according to the first embodiment of the present invention;

FIG. 4 a perspective view illustrating a filter of an infarction prevention device according to a second embodiment of the present invention;

FIGS. 5A to 5C are perspective views illustrating an infarction prevention device according to a third embodiment of the present invention, wherein FIG. 5A schematically illustrates a state when a filter of the infarction prevention device is placed in position inside a blood vessel, FIG. 5B illustrates a portion around a distal end portion of the filter illustrated in FIG. 5A in an enlarged manner, and FIG. 5C illustrates a portion around a base end portion of the filter illustrated in FIG. 5A in an enlarged manner and partially see-through manner; and

FIGS. 6A to 6D are cross-sectional views, illustrating steps for storing, into a sheath tube, the filter of the infarction prevention device according to the third embodiment of the present invention placed inside the brachiocephalic trunk.

### DETAILED DESCRIPTION

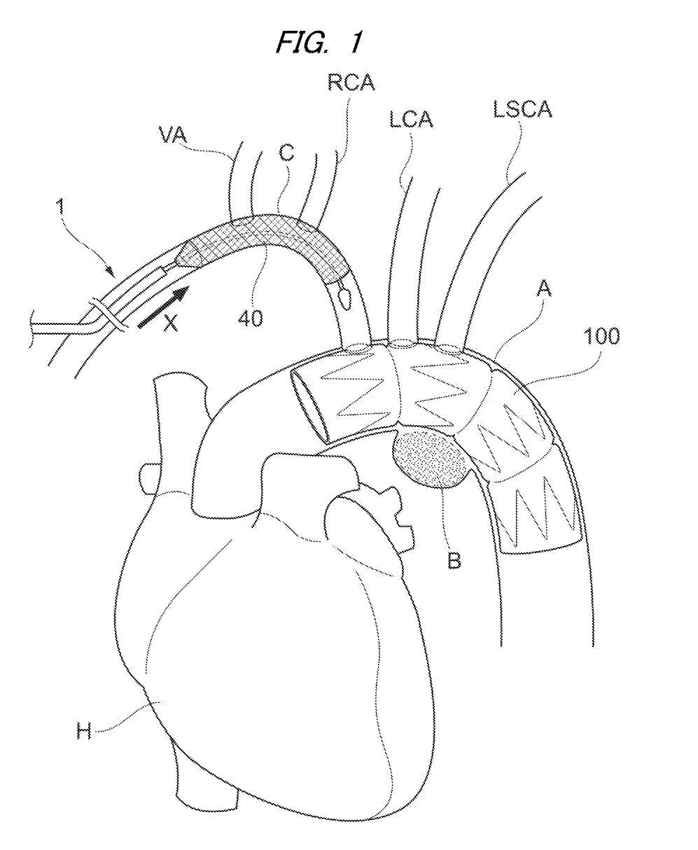

Hereinafter, exemplary embodiments of the present invention will be described in detail with reference to the drawings. First, with reference to FIG. 1, general description will be given with regard to a stent graft used for treatment of an aortic aneurysm and the like and an infarction prevention device used together with the stent graft during the treatment.

As illustrated in FIG. 1, an aorta of the human body is an artery ascending from (left ventricle) of the heart H, curves along an aortic arch A, descends to a branch site at which the abdominal aorta is branched into common iliac arteries, and it is an artery causing blood circulation to the whole body. A state in which a part of a blood vessel wall forming the aorta is swollen in a bump shape is illustrated as an aortic aneurysm B. While not illustrated in the drawings, a state in which peeling (separation) occurs in the media due to blood flowing out through a hiatus or the like occurred in a tunica intima vasorum of the blood vessel wall forming the aorta is called an “aortic dissection”. An aortic aneurysm and the like can occur at various sites of the aorta.

As a surgical treatment of an aortic aneurysm, a method called “stent graft interpolation” is attracting attention in recent years. This method uses a stent graft placing device including a stent graft which is held in a contracted state inside the sheath. Specifically, in this method, for example, a small incision at a base of a thigh called an inguinal region is made, the sheath is inserted into the aorta, and a stent graft is exposed and deployed at the affected area from a distal end of the sheath and placed in the affected area to prevent the rupture of the aortic aneurysm and the like. The stent graft interpolation is a less invasive treatment method that does not require open chest surgery or abdominal surgery as compared to so-called artificial blood vessel replacement surgery. In FIG. 1, a stent graft 100 is placed inside the aortic arch A where the aortic aneurysm B occurred.

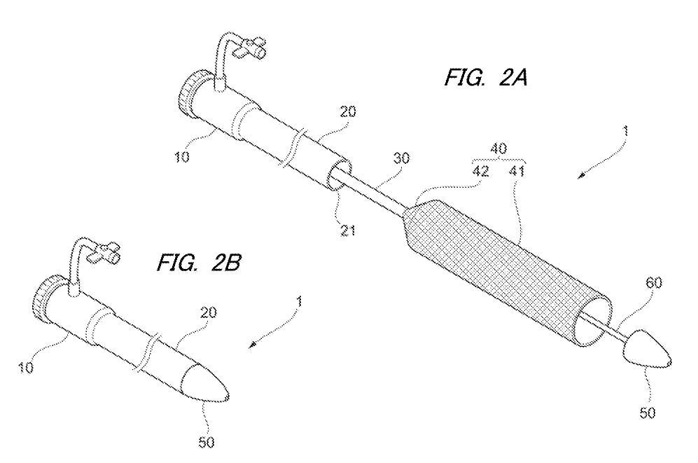

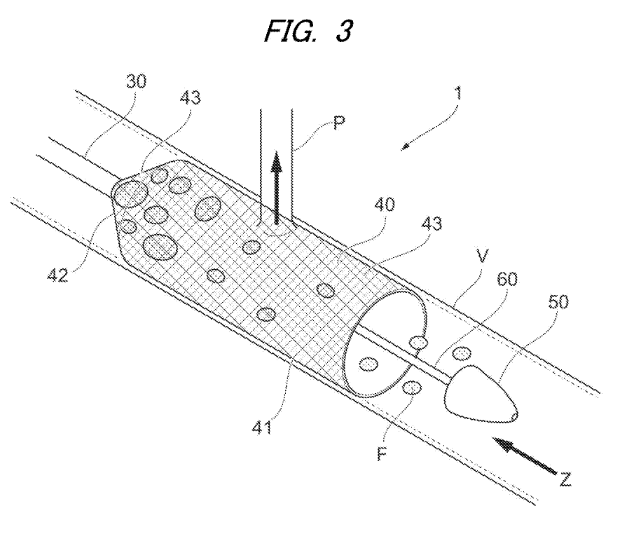

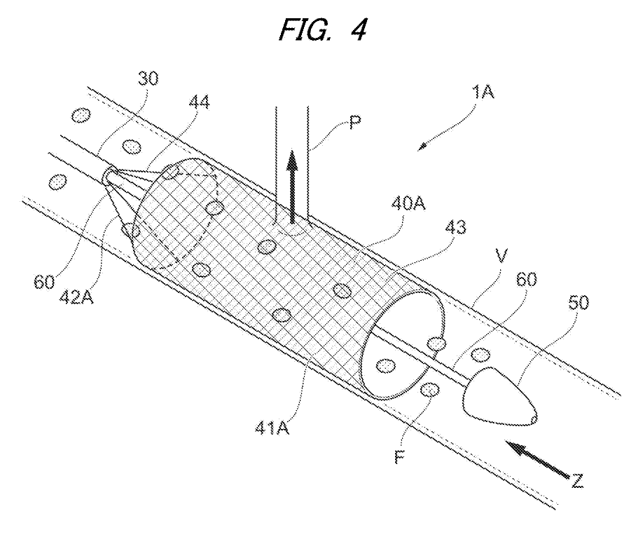

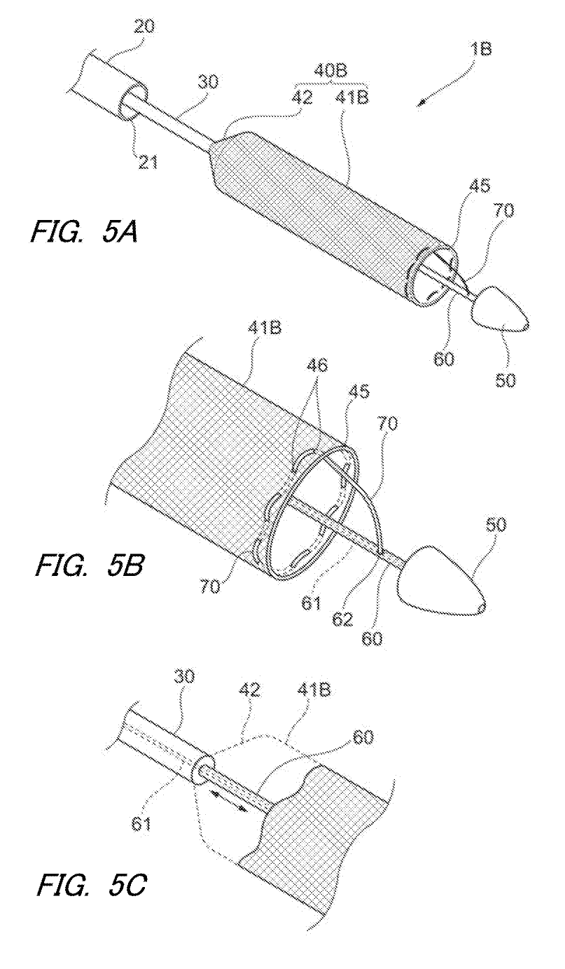

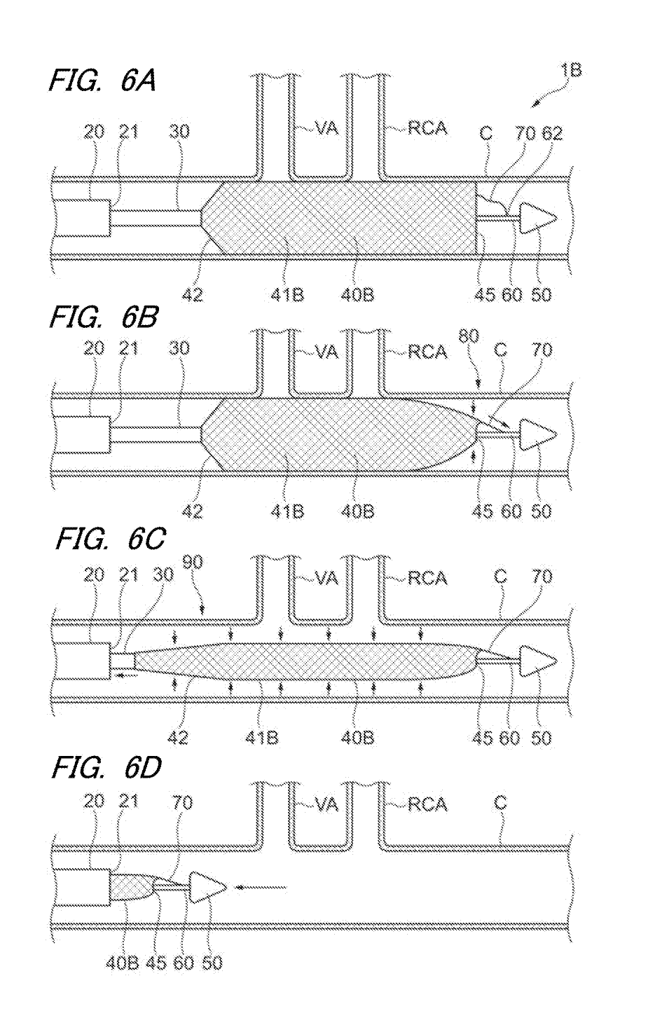

During such treatment, there is a concern that foreign substances F (see, e.g., FIG. 3) such as thrombi are generated and scattered from the aortic arch A (generation source) to cause infarction inside a blood vessel different from the aorta, and therefore, for example, as illustrated in FIG. 1, a filter 40 of an infarction prevention device 1 is temporarily placed in a brachiocephalic trunk C or the like prior to placing the stent graft 100. Accordingly, the filter 40 can prevent the generated foreign substances F such as thrombi from scattering to the right common carotid artery (RCA), vertebral artery (VA), and the like. While FIG. 1 illustrates the infarction prevention device 1 also illustrated in FIGS. 2A to 3, an infarction prevention device 1A illustrated in FIG. 4 and an infarction prevention device 1B illustrated in FIGS. 5A to 6D may also be used in a manner illustrated in FIG. 1.

FIGS. 2A and 2B are perspective views of an infarction prevention device 1 according to a first embodiment of the present invention. FIG. 2A illustrates a state in which the infarction prevention device 1 is placed in position inside a blood vessel, and FIG. 2B illustrates a state in which the infarction prevention device 1 is moved inside the blood vessel. The infarction prevention device 1 includes a main body 10, a sheath tube 20, a filter tube 30 disposed inside the sheath tube 20, a filter 40 attached to a distal end of the filter tube 30, a guide tube 60 disposed inside the filter tube 30, and a tip 50 attached to a distal end of the guide tube 60. When placed inside the blood vessel, a distal end side of the filter 40 is positioned on a central side of the blood vessel, and a base end side of the filter 40 is positioned on a peripheral side of the blood vessel.

The main body 10 is disposed outside the human body, and, for example, a liquid charging port or the like is disposed therein. The sheath tube 20 is attached to a distal end side of the main body 10. The sheath tube 20 is made of, for example, a flexible material. The filter tube 30 is disposed inside the sheath tube 20. The filter tube 30 is made of various materials having suitable hardness and flexibility, such as a resin (plastic, elastomer, or the like) and metal. In addition, the filter tube 30 can move relative to the sheath tube 20 along the longitudinal direction of the sheath tube 20.

The guide tube 60 is disposed inside the filter tube 30. The guide tube 60 can move relative to the filter tube 30 along the longitudinal direction of the filter tube 30. The guide tube 60 is made of, for example, resin, rubber or the like, and inside thereof is hollow and includes a passage through which a guidewire (not illustrated) can be inserted. For example, the guidewire is inserted into a blood vessel in advance, and the guidewire is externally inserted into the guide tube 60 so as to be capable of guiding the infarction prevention device 1 to advance and retreat in the blood vessel as in the state illustrated in FIG. 2B.

The tip 50 is attached to the distal end of the guide tube 60. In order to enable percutaneous insertion operation, for example, the outer diameter of a base end side portion of the tip 50 is substantially equal to the inner diameter of the sheath tube 20, and gradually decreases toward a distal end side. Examples of a material forming the tip 50 include various kinds of materials having suitable hardness and flexibility, such as a synthetic resin (elastomer) composed of a polyamide resin, a polyurethane resin, a polyurethane resin, a polyvinyl chloride resin and the like.

The filter 40 is placed inside a blood vessel such as the brachiocephalic trunk C which is different from the aortic arch A (FIG. 1), and is configured to control the flow of the foreign substances F included in the blood inside the blood vessel. The filter 40 includes a cylindrical body portion (body portion) 41 having a cylindrical outer shape at the time of expansion and a connection portion 42 connected to a base end portion of the cylindrical body portion 41.

The outer diameter of the cylindrical body portion 41 in the expanded state is substantially equal to or greater than an inner diameter of the blood vessel such as the brachiocephalic trunk C, and is brought into contact with the inner wall of the blood vessel in a state of radially pressing the inner wall of the blood vessel outward. A base end side of the connection portion 42 is connected to the distal end of the filter tube 30 (an example of\placing member) by an optional method.

The cylindrical body portion 41 and the connection portion 42 are made of, for example, a resilient material such as metal and resin, and may be made of the same material to or different materials from each other.

As illustrated in FIG. 2B, before being placed in the blood vessel, the filter 40 is stored inside the sheath tube 20 in a radially contracted state. That is, when percutaneously inserting the infarction prevention device 1 into a human body, the infarction prevention device 1 is in a state in which a distal end of the sheath tube 20 and a base end of the tip 50 are in contact with each other. The infarction prevention device 1 is guided by the guidewire to reach the target placement site, and the filter 40 is caused to come out of the sheath tube 20 by backwardly moving the sheath tube 20 toward an operator and away from the filter tube 30 (or by moving the filter tube 30 further inside from the sheath tube 20 when viewed from the operator). When the filter 40 is released from the sheath tube 20, the filter 40, for example, self-expands so as to expand in the radial direction and to be in a state of FIG. 2A.

The method of expanding the filter 40 is an example and is not limited thereto. For example, although a detailed description thereof will be omitted, a known method disclosed in JP2000-350785A or WO2005/099806A1 may be used.

After the use of the filter 40, the sheath tube 20 is moved forward from the filter tube 30 of the infarction prevention device 1 (or the filter tube 30 is moved backward toward the sheath tube 20), whereby the filter 40 is stored inside the sheath tube 20 gradually from its base end to its distal end while contacting an opening edge 21 of the sheath tube 20 at the distal end of the sheath tube 20 and being deformed in a radially contracting manner. Once the filter 40 is fully stored inside the sheath tube 20, the infarction prevention device 1 returns back to the state illustrated in FIG. 2B.

Next, a treatment method for placing the stent graft 100 inside the aortic arch A will be described. In the treatment method, the filter 40 of the infarction prevention device 1 is placed inside the brachiocephalic trunk C, but for example, the filter 40 may be placed in the blood vessel such as the left common carotid artery LCA, the left subclavian artery LSCA (see FIG. 1).

The operator percutaneously inserts the infarction prevention device 1 in which the filter 40 is accommodated in the sheath tube 20 (see FIG. 2B) from the inside of the right elbow (not illustrated) and advances the infarction prevention device 1 from the downstream side of the blood flow to the target placement position of the brachiocephalic trunk C (see arrow X in FIG. 1). Then, the operator moves the sheath tube 20 to the front side with respect to the filter tube 30 of the infarction prevention device 1 to release the filter 40 from the sheath tube 20. The filter 40 is temporarily placed in the brachiocephalic trunk C in a radially expanded state (see FIG. 2A).

Thereafter, the operator places the stent graft 100 in the aortic arch A different from the brachiocephalic trunk C using a known method.

As described above, the filter 40 of the infarction prevention device 1 is placed in the brachiocephalic trunk C when treating the aortic arch A (another blood vessel) different from the brachiocephalic trunk C. After placing the filter 40 in the brachiocephalic trunk C, the aortic arch A (another blood vessel) different from the brachiocephalic trunk C is treated using the stent graft 100 (intravascular treatment tool).

Further, after placing the stent graft 100, the operator moves the sheath tube 20 to the back side with respect to the filter tube 30 of the infarction prevention device 1. Accordingly, the filter 40 is accommodated in the sheath tube 20, and then the entire infarction prevention device 1 is removed from the blood vessel.

FIG. 3 illustrates an example of the filter 40 according to a first embodiment of the present invention. The filter 40 has a configuration that captures foreign substances F such as thrombi at a portion of the filter 40, in particular in the vicinity of the connection portion 42.

Specifically, in the first embodiment, the cylindrical body portion 41 and the connection portion 42 are formed in a meshed manner so that the filter 40 forms a capturing portion 43 capable of capturing the foreign substances F included in the blood inside the blood vessel. Accordingly, not only the cylindrical body portion 41, but also the connection portion 42 can capture the foreign substances F. As illustrated in FIG. 3, the cylindrical body portion 41 disposed along the inner wall of a blood vessel V captures the foreign substances F flowing from the upstream side along the blood flow Z to prevent the foreign substances F from scattering to the side blood vessel P such as the right common carotid artery RCA and the vertebral artery VA illustrated in FIG. 1 and the end artery, the end vein where the foreign substances F cause a trouble when they flow in. At the same time, the connection portion 42 captures the foreign substances F that is not captured by the cylindrical body portion 41. As described above, in the filter 40 of the first embodiment, the connection portion 42 at the most downstream side of the blood flow Z captures the foreign substances F with the cylindrical body portion 41. Therefore, it is possible to control the flow of the foreign substances F, capture the foreign substances F in blood as much as possible to remove the foreign substances F to outside the body.

For example, it is preferable that the connection portion 42 is made of the same material as the material forming the cylindrical body portion 41 and is formed integrally with the cylindrical body portion 41. With such a configuration, the filter 40 can be easily prepared. Further, the connection portion 42, for example, has a conical shape in which the diameter thereof gradually decreases in a direction from the cylindrical body portion 41 side toward the connected portion between the connection portion 42 and the filter tube 30 in a state in which the filter 40 is expanded.

FIG. 4 illustrates a filter 40A of an infarction prevention device 1A according to a second embodiment of the present invention. The filter 40A has a configuration in which at least a portion of the foreign substances F such as thrombi passes through the filter 40A. Specifically, the filter 40A includes the cylindrical body portion 41A formed in a meshed manner to form a capturing portion 43 capable of capturing the foreign substances F included in the blood inside the blood vessel. Accordingly, the filter 40A is configured such that the cylindrical body portion 41A is capable of capturing the foreign substances F but the foreign substances F passes through the connection portion 42 located at the most downstream along the blood flow Z. More specifically, the connection portion 42A includes a plurality of (e.g., four) string members 44 connecting the base end of the cylindrical body portion 41A and the distal end of the filter tube 30.

With this filter 40A of the second embodiment, particularly the cylindrical body portion 41A captures the foreign substances F flowing from the upstream side (central side) along the blood flow Z, and the connection portion 42A on the most downstream (peripheral side) of the blood flow Z is configured such that it is difficult to capture the foreign substances F and allows the foreign substances F to pass through as much as possible. That is, the flow of the foreign substances F is controlled so that the foreign substances F are intentionally caused to flow out in a given direction. According to the configuration, following advantages are obtained. Scattering of the foreign substances F can be suppressed when the foreign substances F being collected by the filter 40A, and clogging of the filter 40A is prevented even when there are many foreign substances F.

In the second embodiment, a direction in which the foreign substances F is caused to flow out is not particularly limited. However, the direction is basically not a direction toward the side blood vessel P such as the right common carotid artery RCA, the vertebral artery VA, the end artery, the end vein where the foreign substances F cause a trouble when they flow in, but an example of the direction includes a direction toward a peripheral blood vessel and the like.

Further, the structure of the connection portion 42A is not limited to the string members 44, and for example, the connection portion 42A may be configured in a meshed manner but with rougher mesh than the mesh of the cylindrical body portion 41A. The area of the mesh of the connection portion 42A and the size of the space area between the adjacent string members 44 are adjusted to be capable of controlling the amount of the outflow or the amount of capture of the foreign substances F.

As described above, by using the infarction prevention device 1, 1A according to the first and second embodiments of the present invention in an intravascular treatment such as a stent graft interpolation to treat a site where infarction may occur due to the foreign substances F, even if the foreign substances F generated in a site (e.g, the aortic arch A) different from the blood vessel in which the filter 40, 40A of the infarction prevention device 1, 1A is placed is scattered, it is possible to control the flow of the foreign substances F included in the blood inside the blood vessel where the infarction prevention device 1, 1A is placed. Therefore, it is possible to prevent the foreign substances F from flowing into the lateral blood vessels, particularly the end artery and end vein not connected to other arteries and veins, and to appropriately prevent the onset of cerebral infarction (tissue necrosis).

In addition, since the filter 40, 40A elongates when contracting and has a mesh structure in which the foreign substances F is entangled, the filter 40 can collect the foreign substances F which is larger than the size of an IVC (Inferior Vena Cava) filter of the related art without scattering the foreign substances F. In addition, unlike the protection filter for a carotid artery stent, since it is possible to place the infarction prevention device 1 from the downstream side of the blood flow of the blood vessel where the filter 40, 40A is to be placed, another member for intravascular treatment which accesses from the upstream side of the blood flow can also be used in combination.

FIGS. 5A to 6D illustrate an infarction prevention device 1B according to a third embodiment of the present invention. The infarction prevention device 1B is configured to include a filter closing mechanism 80 capable of closing a distal end opening of the cylindrical body portion 41B of the filter 40B, and a filter contracting mechanism 90 for contracting the entire cylindrical body portion 41B of the filter 40B radially inward. Here, “radially inward” is, in other words, in a direction away from the inner wall of the blood vessel.

Specifically, as illustrated in FIGS. 5A and 5B, the infarction prevention device 1B according to the third embodiment is configured to include, in addition to the elements similar to those of the infarction prevention device 1 according to the first embodiment, a closure string 70 serving as part of the filter closing mechanism 80 and the filter contracting mechanism 90.

The closure string 70 is provided to extend inside a hollow portion 61 (lumen) of the guide tube 60. One end of the closure string 70 extends out of the hollow portion 61 from an opening 62 formed at a distal end portion of the guide tube 60, whereas the other end of the closure string 70 extends out of the hollow portion 61 from an opening (omitted from the drawings) formed at a given location on the main body 10. A portion of the closure string 70 exposed to the outside from the opening 62 is inserted sequentially into a plurality of holes 46 in a circumferential direction in a stitching manner, the holes 46 being formed near the distal end opening 45 of the cylindrical body portion 41B at an interval in the circumferential direction. A terminal end of the portion of the closure sting 70 inserted into the holes 46 is fixed at a given location on the cylindrical body portion 41 in an optional way. The opening 62 of the guide tube 60 is located on the distal side than the distal end opening 45 of the filter 40B.

Hereinafter, the filter closing mechanism 80 and the filter contracting mechanism 90 will be described in more detail with reference to FIGS. 6A to 6D. FIGS. 6A to 6D illustrate an exemplary steps for storing the used filter 40B in the sheath tube 20 from a state in which the cylindrical body portion 41B of the filter 40B is placed inside the brachiocephalic trunk C illustrated in FIG. 1.

As illustrated in FIG. 6A, in a state in which the filter 40B is placed inside the brachiocephalic trunk C, the cylindrical body portion 41B of the expanded filter 40 covers the branch corresponding to the right common carotid artery RCA and the vertebral artery VA. The foreign substances F (see, FIG. 3) is captured inside the expanded filter 40B.

From this state, in order to store the filter 40 inside the sheath tube 20, first, as illustrated in FIG. 6B, the operator draws the other end (not illustrated) of the closure string 70 led out of the hollow portion 61 on a side of the main body 10 of the infarction prevention device 1B toward the operator (to the left in FIG. 6B). As shown in FIG. 6B, this causes the closure string 70 connected to a portion near the distal end opening 45 of the cylindrical body portion 41B to be pulled toward the guide tube 60 so that a force is applied to the cylindrical body portion 41B to close the distal end opening 45. As a result, the distal end opening 45 of the cylindrical body portion 41 moves radially inward, away from the inner wall of the brachiocephalic trunk C. In other words, the cylindrical body portion 41 is deformed such that the distal end opening 45 of the cylindrical body portion 41 is closed. This prevents the foreign substances F captured inside the filter 40B to be dispersed outside the filter 40B again through the distal end opening 45 of the cylindrical body portion 41B. In this manner, the hollow portion 61 and the opening 62 of the guide tube 60 and the closure string 70 together provide the filter closing mechanism 80.

The distal end opening 45 of the cylindrical body portion 41B being “closed” means that the cylindrical body portion 41B is deformed such that an opening area of the distal end opening 45 is reduced. Specifically, the cylindrical body portion 41B may be deformed such that the opening area of the distal end opening 45 becomes substantially zero. Alternatively, the cylindrical body portion 41B may be deformed such that the opening area of the distal end opening 45 becomes smaller than the state illustrated in FIG. 6A but larger than zero.

Next, in a state in which the distal end opening 45 of the cylindrical body portion 41B is closed, the operator fixes the other end of the closure string 70 on the side of the main body 10, so that the position of the distal end opening 45 of the cylindrical body portion 41B to which the closure string 70 is connected is held so as not to shift in the axial direction. Then, as illustrated in FIG. 6C, the operator pulls the filter tube 30 toward the operator (to the left in FIG. 6C) relative to the sheath tube 20 and the guide tube 60. This causes the cylindrical body portion 41B to be pulled toward the base end side in a state in which the position of the distal end opening 45 is kept by the closure string 70, so that a force is applied to the filter 40B such that the entire filter 40B extends in the axial direction. At the same time, the entire filter 40B deforms radially inward away from the inner wall of the brachiocephalic trunk C, so that the cylindrical body portion 41B is separated from the inner wall of the brachiocephalic trunk C. In other words, the entire filter 40 is deformed so as to contract radially inward. Here, the filter tube 30, the guide tube 60 and the closure string 70 together provide the filter contracting mechanism 90.

Next, as illustrated in FIG. 6D, while maintaining the relative position between the filter tube 30 and the guide tube 60 (that is, while maintaining the contraction state of the filter 40B) the operator pulls the filter tube 30 and the guide tube 60 toward the operator (to the left in FIG. 6D) relative to the sheath tube 20 so that the filter 40B is stored inside the sheath tube 20.

By storing the filter 40B inside the sheath tube 20 with the filter 40B being held in the contracted state, the filter 40B is suppressed from contacting an opening edge of the distal end opening 21 of the sheath tube 20. Thus, even when the foreign substances F partially protrude out from the meshes of the filter 40B, the portion of the foreign substances F is prevented from contacting the opening edge of the distal end opening 21 of the sheath tube 20 and from dispersing in the blood. That is, the foreign substances F captured inside the filter 40B is prevented from dispersing again at the time of storing the filter 40B.

Further, in the example illustrated in FIGS. 6A to 6D, over the entire procedure of FIGS. 6A to 6D, the tip 50 (a distal end portion of the infarction prevention device 1) does not move from the position illustrated in FIG. 6A to the central side (the right side in FIGS. 6A to 6D) of brachiocephalic trunk C. Therefore, the tip 50 is prevented from contacting the stent graft 100 placed in the aortic arch A on the central side of the brachiocephalic trunk C.

Further, as illustrated in FIG. 6D, the filter 40B is stored in the sheath tube 20 in the brachiocephalic trunk C and at a position downstream from the branch of the vertebral artery VA. Therefore, even if a portion of the foreign substances F protruding out of the meshes of the filter 40B it is prevented from flowing into the right common carotid artery RCA or to the vertebral artery VA.

In the embodiments described above, when placing the filter 40, 40A, 40B of the infarction prevention device 1, 1A, 1B inside the left common carotid artery LCA and the left subclavian artery LSCA, the cylindrical body portion 41, 41A, 41B may not necessarily be configured as the capturing portion 43, and at least the connection portion 42 of the filter 40, 40A, 40B may be configured as the capturing portion 43.

When placing the filter 40, 40A, 40B of the infarction prevention device 1, 1A, 1B inside the brachiocephalic trunk C, the entire cylindrical body portion 41, 41A, 41B of the filter 40, 40A, 40B may not necessarily form the capturing portion 43, and only a portion of the cylindrical body portion 41, 41A, 41B corresponding to at least the lateral blood vessel P such as the right common carotid artery RCA and the vertebral artery VA, the end artery, the end vein and the like where it is undesirable to have the foreign substances F flow therein may be formed in a meshed manner and form the capturing portion 43 capable of capturing the foreign substances F included in the blood inside the blood vessel.

In the embodiments described above, the generation source of the foreign substances F has been described as a blood vessel different from the blood vessel (e.g., the brachiocephalic trunk C) inside which the filter 40, 40A, 40B is placed, but this is merely an example, and it may be a site inside the blood same vessel where the filter 40, 40A, 40B is placed but different from the site at which the filter 40, 40A, 40B is placed, or may be both the different blood vessel and the same blood vessel.

The filter 40, 40A, 40B may have any configuration in so far as it can capture the foreign substances F included in the blood inside the blood vessel using the capturing portion 43, and control a flow of the foreign substances included in the blood inside the blood vessel. That is, the capturing portion 43 need not be formed in a meshed manner, and may for example be formed, although not illustrated in the drawings, by bending thin metal wires in a zigzag shape or a wavy shape such that mountain portions and valley portions are alternately formed.

In the above embodiment, when treating the aortic aneurysm B formed in the aortic arch A, the filter 40, 40A, 40B of the infarction prevention device 1, 1A, 1B is placed in the blood vessel such as the brachiocephalic trunk C, the left common carotid artery LCA, the left subclavian artery LSCA or the like, but it is merely an example and the present invention is not limited thereto. The blood vessel in which the filter 40, 40A, 40B is placed can be optionally changed. For example, when treating an abdominal aortic aneurysm (not illustrated), the filter 40, 40A, 40B of the infarction prevention device 1, 1A, 1B may be placed in the left and right renal arteries to prevent the onset of renal infarction. In addition, for example, when treating a blood vessel such as the brachiocephalic trunk C, the left common carotid artery LCA, the left subclavian artery LSCA, or the like, the infarction prevention device 1 may be placed in the blood vessel.

That is, the infarction prevention device 1, 1A, 1B can effectively control the flow of various foreign substances F of various kinds and sizes.

In the third embodiment described above, the entire filter 40B is caused to contract (see FIGS. 6B and 6C) by pulling the base end portion of the filter 40B using the filter tube 30 in a state in which the position of the distal end portion of the filter 40B is held by the closure string 70. However, to cause the entire filter 40B to contract, the force for extending the filter 40B in the axial direction may be applied to the filter 40B through other steps and/or configuration. That is, in so far as the filter 40B is deformed such that the distance between the base end portion of the distal end portion of the filter 40B becomes longer, for example, the distal end portion may be moved further toward the distal side while fixing the base end portion, the base end portion may be moved further toward the base side while fixing the distal end portion, or both the distal end portion and the base end portion may be moved such that they are separated away from each other.

Further, in the third embodiment described above, the position of the sheath tube 20 is fixed and then the contracted filter 40B is pulled into the sheath tube 20 to store the filter 40B inside the sheath tube 20 (see FIG. 6D). However, a method for storing the filter 40B is not limited to this example. For example, the filter 40B may be stored by pushing the sheath tube 20 forward with the contracted filter 40B being fixed, or by moving the sheath tube 20 forward and at the same time pulling the filter 40B into the sheath tube 20. Further, from the standpoint of more suitably preventing the foreign substances F from flowing into the right common carotid artery RCA and the vertebral artery VA, the filter 40B may be stored inside the sheath tube 20 after moving the entire infarction prevention device 1B, including the contracted filter 40B and the sheath tube 20, to downstream of the branching point between right common carotid artery RCA and the vertebral artery VA relative to the blood flow (to the left in FIG. 6D).

Further, in the third embodiment described above, the closure string 70 is inserted into a plurality of holes 46 formed near the distal end opening 45 of the cylindrical body portion 41B. However, the closure string 70 may be attached to the cylindrical body portion 41B in any other way in so far as it can close the distal end opening 45 of the cylindrical body portion 41B. For example, the closure string 70 may be inserted through a tubular passage provided to extend along a circumferential direction near the distal end opening 45 of the cylindrical body portion 41B, or may be provided in a spiral manner near the distal end opening 45 of the cylindrical body portion 41B, or may be provided in a zigzag manner along the circumferential direction. Depending of the structure of the cylindrical body portion 41B, the closure string 70 need not always apply external force over the entire circumference of the distal end opening 45 like in the third embodiment, and instead the closure string 70 may apply external force only to a portion of the circumference of the distal end opening 45.

In the third embodiment described above, the closure string 70 is provided in the filter 40 of the infarction prevention device 1 according to the first embodiment (see FIG. 3), but the closure string 70 may be provided in the filter 40A of the infarction prevention device 1A according to the second embodiment (see FIG. 4).

While at least one of the cylindrical body portion 41, 41A, 41B and the connection portion 42 of the filter 40, 40A, 40B is formed in a meshed manner, this is merely a non-limiting example, and may be modified in an optional manner in so far as it is configured to have pores allowing passage of the blood. For example, it may have a woven structure of wefts and warps interlaced with each other, or a laser perforated structure.

The present invention is not limited to the above-described embodiment, but may be appropriately modified, improved. In addition, the material, shape, size, numerical value, form, number, placement site, and the like of each constituent element in the embodiment described above are optional as long as it can achieve the present invention, and are not limited.

The infarction prevention device of the present invention can effectively control the flow of various foreign substances that can occur during a treatment of a blood vessel and is useful for suitably preventing the onset of infarction.

What is claimed is:

1. An infarction prevention device comprising a filter to be placed inside a blood vessel, wherein the filter comprises:
a connection portion having a base end to be connected to a placing member configured to place the filter inside the blood vessel; and
a body portion having a cylindrical outer shape and connected to a front end of the connection portion, and
wherein at least one of the connection portion and the body portion comprises a capturing portion configured to capture a foreign substance included in blood inside the blood vessel such that the at least one of the connection portion and the body portion controls a flow of the foreign substance included in the blood inside the blood vessel.

2. The infarction prevention device according to claim 1, wherein the capturing portion is configured to capture the foreign substance included in the blood, a generation source of the foreign substance being at a site different from a site where the filter is to be placed.

3. The infarction prevention device according to claim 1, wherein the connection portion and the body portion comprise the capturing portion having pores that allow passage of the blood.

4. The infarction prevention device according to claim 1, wherein the body portion comprises the capturing portion having pores that allow passage of the blood, and
wherein the connection portion is configured to allow passage of the foreign substance included in the blood inside the blood vessel.

5. The infarction prevention device according to claim 1, further comprising a configuration to close a distal end opening of the body portion, the distal end opening being provided to be positioned on a central side of the blood vessel when the filter is placed inside the blood vessel.

6. The infarction prevention device according to claim 1, further comprising a configuration to contract the body portion of the filter placed inside the blood vessel in a direction away from an inner wall of the blood vessel.

7. The infarction prevention device according to claim 1, wherein the filter is configured to be placed inside the blood vessel when treating another blood vessel.

8. A treatment method comprising placing the filter of the infarction prevention device according to claim 1 inside the blood vessel, and after the placing, treating another blood vessel different from the blood vessel using an intravascular treatment tool.
